(19) United States
(12) Patent Application Publication
(10) Pub. No.: US 2005/0176804 A1
(43) Pub. Date: Aug. 11, 2005

(54) Method and material for free radical scavenging in human tissue

(76) Inventor: Jin Song, Dallas, TX (US)

Correspondence Address: CARSTENS YEE & CAHOON, LLP, P O BOX 802334, DALLAS, TX, 75380, US

(21) Appl. No.: 10/982,448

(22) Filed: Nov. 4, 2004

Related U.S. Application Data
(60) Provisional application No. 60/517,285, filed on Nov. 4, 2003.

(51) Int. Cl. A61K 31/405

(52) U.S. Cl. 514/419

(57) ABSTRACT

The present invention provides agents that reduce free-radical activity in skin cells. The agents can include Indole derivatives such as Indoleacetyl phenylalanine and can be applied to the skin topically. The agents reduce free-radical activity both by direct scavenging of free radicals and increasing production of antioxidant enzymes, as well as reducing production of pro-oxidant enzymes such as nitric oxide synthase.

### CROSS REFERENCE TO RELATED APPLICATIONS

This application claims the benefit of and priority to a U.S. Provisional Patent Application No. 60/517,285 filed Nov. 4, 2003, the technical disclosure of which is hereby incorporated herein by reference.

### BACKGROUND OF THE INVENTION

1. Technical Field

The present invention relates generally to the use of antioxidant compounds in cosmetic preparations, and more specifically the use of indoles as antioxidant agents.

2. Description of Related Art

Human skin is subject to deterioration due to a variety of factors including deleterious free radicals and oxidants generated from external sources such as ultraviolet (UV) radiation from sunlight, and air pollutants, as well as from natural oxidative processes occurring within skin cells themselves.

Typically, stable molecules contain paired electrons (i.e. even number). Certain chemical reactions can break the bonds that hold paired electrons together, resulting in the formation of free radicals, which are molecules that contain an odd number of electrons, making them unstable, short-lived, and highly reactive. To gain stability, a free radical needs to gain an additional electron in order to return to an even number of electrons. Free radicals acquire this additional electron by reacting with the nearest atom or molecule. As the free radical combines with other atoms and molecules, new radicals are created, and a chain reaction begins. In the living tissue, free radicals are believed to cause tissue damage at the cellular level, e.g., DNA, mitochondria, cell membrane, etc.

Antioxidants are molecules that are capable of donating electrons to free radicals without becoming free radicals themselves. Thus, antioxidants stop the chain reaction and prevent further damage to cell structures. Examples of antioxidants include nutritional compounds, e.g., vitamins E and C, selenium, and anthocyanadins, as well as enzymes produced within cells, e.g., Superoxide Dismutase (SOD).

In the specific case of skin cells, oxidative damage from free radicals produces changes to the skin structure and texture, including the appearance of wrinkles in the skin. Many cosmetic and skin care products aimed at preserving and restoring skin health employ topical antioxidant compounds to reduce the activity of free radicals in skin cells.

A class of chemical compounds known as indoles includes derivative particularly noted for antioxidant activity. The indole nucleus, known to chemists as benzopyrrole, is the parent member of a broad spectrum of nitrogen heterocyclic biochemicals commonly found in nature. Indoles also exist as melanin-related organics and indigoid pigments, making certain indole derivatives uniquely suited for use as antioxidant agents in cosmetic skin products.

### SUMMARY OF THE INVENTION

The present invention provides agents that reduce free-radical activity in various tissue cells. The agents include Indole derivatives, especially Indoleacetyl phenylalanine, and can be applied to the skin topically. The agents reduce free-radical activity both by direct scavenging of free radicals and increasing production of antioxidant enzymes, as well as reducing production of pro-oxidant enzymes such as nitric oxide synthase.

### DETAILED DESCRIPTION OF THE INVENTION

A great deal of research has focused on how to keep skin looking young. Many creams have been developed to help sooth fine wrinkles so both men and women appear more youthful. Sometimes the improvement in appearance is obtained by simply making the skin temporarily contract, and thus appear taut. However, the true cause of wrinkles is deterioration in the cell structure of the connective tissue under the epidermis, which is often the result of oxidative damage from free radicals.

The present invention utilizes indole derivatives as active antioxidant ingredients in topical cosmetic preparations or in oral preparations (i.e. pill, capsule). The indole derivatives were selected based on empirical evidence of their ability to suppress harmful reactive species from both oxygen- and nitrogen-derived free radicals and oxidants.

Specifically, indoleacetyl phenylalanine has remarkable actions against oxidative stress including, 1) suppression of various reactive oxygen species (ROS) and reactive nitrogen species (RNS) as demonstrated in skin fibroblasts, 2) up-regulation of two major anti-oxidative scavenger enzymes, Superoxide Dismutase (SOD) and catalase, and 3) boosting of Redox balance.

The anti-oxidative action of indoleacetyl phenylalanine was tested against reactive oxygen species (ROS) using human skin fibroblast cultures. Total ROS generation in skin fibroblasts was measured using the fluorescent dye dichlorofluorescin diacetate (DCFDA) as a probe. DCFDA readily diffuses through cell membranes and is enzymatically hydrolyzed by intracellular esterase to become non-fluorescent dichlorofluorescin (DCFH), which is then oxidized by reactive species (oxidants) to highly fluorescent dichlorofluorescin (DCF). Increased antioxidant activity within a cell is marked by a reduction in the fluorescent DCF.

Using this fluorescent marking technique, indoleacetyl phenylalanine demonstrated significant antioxidant activity toward ROS generation in skin fibroblasts without affecting cell viability. Positive results were obtained with fibroblasts incubated in both serum-containing and serum-free media (serum proteins, e.g., albumin, can act as antioxidants and skew test results). In 10% serum media, indoleacetyl phenylalanine exhibited significant antioxidant activity at concentrations of 15, 30, and 60 μM. In serum-free media, indoleacetyl phenylalanine exhibited antioxidant activity at concentrations of 7.5, 15, and 30 μM.

Indoleacetyl phenylalanine also showed antioxidant activity specifically against t-butyl hydroperoxide (t-BHP) induced ROS activity in serum-free media in a dose dependent manner.

In addition to ROS activity, indoleacetyl phenylalanine was also tested against radical nitrogen species (RNS) in cultured human skin fibroblasts. To estimate suppression of RNS, total nitrate plus nitrite (Nox) generation was measured using the Greiss assay method. At a concentration of 60 μg/ml, indoleacetyl phenylalanine decreased RNS generation by 33% as compared to an untreated control. However, it should be noted that the inhibition of RNS generation requires higher concentrations, suggesting that indoleacetyl phenylalanine may not be as effective for RNS as for ROS.

The antioxidant effects of indoleacetyl phenylalanine was also tested with respect to the production of antioxidant enzymes by means of differential gene expression in human skin fibroblasts. The experiment was conducted with respect to gene expression related to two major defense enzymes, Superoxide Dismutase (SOD) and catalase. Using the Western blotting method, experimental data showed that indoleacetyl phenylalanine increased gene expression for the production of both Cu/ZnSOD and catalase based on their protein amounts. Gene expression for both enzymes was stimulated at indoleacetyl phenylalanine concentrations of 15 and 30 μM. Increased production of catalase was particularly increased at 30 μM. These results are particularly remarkable because they demonstrate that indoleacetyl phenylalanine is unique in that it acts not only as an antioxidant but also a stimulant to increase production of antioxidant enzymes, thus giving it both direct and indirect antioxidant benefits.

Following previous leads on indoleacetyl phenylalanine's finding as a potent anti-free radical substance, additional experimental evidence was sought to strengthen the earlier findings. Focus at this time was placed on nitric oxide synthase (iNOS), which is the key enzyme for generating a destructive oxidant, nitric oxide. Since this enzyme existed in native and activated states, it was pertinent to check both the enzyme activities to ascertain the inhibitory action of indoleacetyl phenylalanine. The results on both enzyme statuses are all positive in that indoleacetyl phenylalanine at very low levels (3-12 μM) suppressed iNOS for native and activated states. The more impressive aspect of these findings about the potency of indoleacetyl phenylalanine is that even in activated state elicited by 5 μM t-BHP, indoleacetyl phenylalanine is able to suppress iNOS gene expression activity.

Therefore, putting together on all of the above findings, indoleacetyl phenylalanine has the unique ability to reduce oxidative stress not only through direct scavenging of different free radicals but also by inhibiting oxidant-generating enzymes, which is uncommon in most known antioxidants.

As shown above, the antioxidant effects of indoleacetyl phenylalanine occur at very low concentrations. In addition to antioxidant activity, indoleacetyl phenylalanine exhibits anti-inflammatory effects through inhibition of fibroblast COX-2 enzymes. Indole derivative can also stimulate collagen synthesis with as little as 3 μM.

Furthermore, to investigate the effects of indole derivatives on cell proliferation as a biological marker, cell viability was assayed using the MTT method. Of the indole derivatives tested, indoleacetyl phenylalanine produced the most active response by stimulating cell proliferation by 30% as compared to untreated controls, indicating protection against peroxidative insult under experimental conditions. Further experimental results indicate that during a four-day incubation period, the fibroblasts were effectively proliferated in a dose-dependent manner with as little as 2 μM indoleacetyl phenylalanine.

Indole derivative have also been shown to promote tissue wound healing by promoting the migration of fibroblast into damaged cellular areas. This effect can be achieved from 0.10 ppm to as high at 250 ppm.

The low concentrations used to achieve the above results are of great advantage, as some indole derivatives can inhibit fibroblast cell growth at high concentrations. By achieving significant antioxidant activity, as well as anti-inflammatory effects, collagen synthesis, cell proliferation and cell migration at concentrations of 250 ppm and lower, the indole derivative of present invention can protect and repair skin cells without interfering with normal fibroblast cell growth.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated.

1. A method of scavenging free-radicals in tissue, the method comprising delivering a therapeutically effective dosage of indole derivatives, wherein said dosage reduces free-radical activity in skin cells.

2. The method according to claim 1, wherein said step of delivering a therapeutically effective dosage comprises delivering indoleacetyl phenylalanine.

3. The method according to claim 1, wherein said dosage stimulates the increased endogenous production of antioxidant enzymes.

4. The method according to claim 3, wherein said dosage increases endogenous production of Superoxide Dismutase.

5. The method according to claim 3, wherein said dosage increases endogenous production of catalase.

6. The method according to claim 1, wherein said dosage reduces endogenous production of nitric oxide synthase.

7. The method according to claim 1, wherein said dosage reduces the activity of reactive oxygen species.

8. The method according to claim 1, wherein said dosage reduces the activity of reactive nitrogen species.

9. The method according to claim 1, wherein the step of delivering comprises topical application.

10. The method according to claim 1, wherein the step of delivering comprises oral administration.

11. The method according to claim 1, wherein said therapeutic dosage is between 0.10 ppm and 250 ppm.

12. Indole derivatives delivered in a therapeutically effective dosage that reduces free-radical activity in skin cells.

13. The indole derivatives according to claim 12, wherein said indole derivatives comprise indoleacetyl phenylalanine.

14. The indole derivatives according to claim 12, wherein said dosage stimulates the increased endogenous production of antioxidant enzymes.
